(19) United States
(12) Patent Application Publication
(10) Pub. No.: US 2018/0050160 A1
(43) Pub. Date: Feb. 22, 2018

(54) Drug Delivery Device

(71) Applicant: Sanofi, Paris (FR)

(72) Inventors: Simon Lewis Bilton, Warwickshire (GB); Matthew Meredith Jones, Warwick, Warwickshire (GB); William Marsh, Buckinghamshire (GB); Anthony Paul Morris, Coventry West Midlands (GB)

(21) Appl. No.: 15/516,523

(22) PCT Filed: Oct. 9, 2015
(86) PCT No.: PCT/EP2015/073420

§ 371 Date: Apr. 3, 2017

(30) Foreign Application Priority Data
Oct. 9, 2014 (EP) 14306586.0

(51) Int. Cl. A61M 5/315; A61M 5/20; A61M 5/24

(52) U.S. Cl. CPC A61M 5/31553; A61M 5/20; A61M 2005/3126; A61M 2005/2026; A61M 2005/2407; A61M 5/24

(57) ABSTRACT

The disclosure refers to an injection device for automatic spring driven injection of a liquid drug by which doses of an individual size can be set by a user. The injection device comprises a housing defining an interior space and having a longitudinal window, a rotatable dose dial axially retained in relation to the housing, a rotatable scale drum carrying indicia for indicating the size of the set dose, wherein the scale drum is functionally coupled to the dose dial to rotate when the dose dial is rotated to set a dose, a sliding element provided with a sliding window, which sliding element is adapted to slide axially in relation to the housing during dose setting, and through which sliding window the indicia carried by the scale drum is visible. The longitudinal window and the sliding window in combination with the indicia form a dose size display. The scale drum rotates within the interior space defined by the housing during dose setting. The inner surface of the sliding element is provided with an internal feature engaging an external thread provided on the outer surface of the scale drum The sliding element is further axially guided in the housing such that the sliding element moves axially when the scale drum is rotated. A drive spring is attached to the scale drum with one end and to the housing with another end such that relative rotation between the scale drum and the housing charges the drive spring.

### CROSS-REFERENCE TO RELATED APPLICATIONS

This application is a U.S. national stage application under 35 USC §371 of International Application No. PCT/EP2015/073420, filed on Oct. 9, 2015, which claims priority to European Patent Application No. 14306586.0 filed on Oct. 9, 2014, the entire contents of which are incorporated herein by reference.

### TECHNICAL FIELD

The present disclosure is generally directed to an injection device, i.e. a drug delivery device for automatic spring driven injection of a liquid drug, i.e. a medicament, by which doses of an individual size can be set by a user.

### BACKGROUND

Drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (i.e., reusable) and non-resettable (i.e., disposable). For example, disposable drug delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. Some embodiments are applicable for both types of devices, i.e. for disposable devices as well as for reusable devices.

A further differentiation of drug delivery device types refers to the drive mechanism: There are devices which are manually driven, e.g. by a user applying a force to an injection button, devices which are driven by a spring or the like and devices which combine these two concepts, i.e. spring assisted devices which still require a user to exert an injection force. The spring-type devices involve springs which are preloaded and springs which are loaded by the user during dose selecting. Some stored-energy devices use a combination of spring preload and additional energy provided by the user, for example during dose setting. Further types of energy storage may comprise compressed fluids or electrically driven devices with a battery or the like. Although many aspects are applicable for all of these types of devices, i.e. for devices with or without a drive spring or the like energy storage, the preferred embodiments require some kind of energy storage. These types of delivery devices generally comprise three primary elements: a cartridge section that includes a cartridge often contained within a housing or body or holder; a needle assembly connected to one end of the cartridge section; and a dosing section connected to the other end of the cartridge section. A cartridge (often referred to as an ampoule) typically includes a reservoir that is filled with a medication (e.g., insulin), a movable rubber type bung or stopper located at one end of the cartridge reservoir, and a top having a pierceable rubber seal located at the other, often necked-down, end. A crimped annular metal band is typically used to hold the rubber seal in place. While the cartridge housing may be typically made of plastic, cartridge reservoirs have historically been made of glass.

The needle assembly is typically a replaceable double-ended needle assembly. Before an injection, a replaceable double-ended needle assembly is attached to one end of the cartridge assembly, a dose is set, and then the set dose is administered. Such removable needle assemblies may be threaded onto, or pushed (i.e., snapped) onto the pierceable seal end of the cartridge assembly.

The dosing section or dose setting mechanism is typically the portion of the device that is used to set (select) a dose. During an injection, a lead screw, a plunger or piston rod contained within the dose setting mechanism presses against the bung or stopper or piston of the cartridge. This force causes the medication contained within the cartridge to be injected through an attached needle assembly. After an injection, as generally recommended by most drug delivery device and/or needle assembly manufacturers and suppliers, the needle assembly is removed and discarded.

The dosing section of drug delivery devices for selecting and dispensing a number of user variable doses of a medicament often comprises a display for indicating the selected dose to a user. This is especially important where a user may select a different dose each time depending on the state of health. There are mechanical displays, e.g. a drum with printed numbers on its outer surface, wherein the number corresponding to the actually selected dose is visible through a window or opening in the device. Although such mechanical displays are simple and reliable, they usually require a relatively large construction space which makes the devices bulky.

### SUMMARY

Advantages of some embodiments may provide an injection device requiring low actuation forces and may additionally. reduce the number of components of the injection device needed to make the injection device economically efficient.

According to a first embodiment, the injection device comprises a housing defining an interior space and having a longitudinal window, a rotatable dose dial axially retained in relation to the housing, a rotatable scale drum carrying indicia for indicating the size of the set dose, wherein the scale drum is functionally coupled to the dose dial to rotate when the dose dial is rotated to set a dose, a sliding element provided with a sliding window, wherein the sliding element is adapted to slide axially in relation to the housing during dose setting, and through which sliding window the indicia carried by the scale drum is visible such that the longitudinal window and the sliding window in combination with the indicia form the dose size display, and wherein the rotatable scale drum rotates within the interior space defined by the housing during dose setting wherein the inner surface of sliding element is provided with an internal feature engaging an external thread provided on the outer surface of the scale drum and wherein the sliding element is axially guided in the housing such that the sliding element moves axially when the scale drum is rotated and wherein a drive spring is attached to the scale drum with one end and to the housing with another end such that relative rotation between the scale drum and the housing charges the drive spring.

In some aspects the injection device reduces the number of parts of the injection device and generates less friction.

The dose dial may be configured as a dial grip for setting user variable doses of a medicament. The drive spring is charged by rotation of the dose drum scale and the energy stored in the drive spring during said charging is sufficient to provide the energy necessary to move a lead screw or the like in distal direction so as to drive a bung in a cartridge in the distal direction such that medicament is dispensed from the cartridge. The dose scale drum may be configured as a sleeve-like component, e.g. a number sleeve. The sliding element may be configured as a gauge component with an aperture or window, wherein the position of the gauge component is used to identify the set and/or dispensed dose. The combination of scale drum, sliding window and longitudinal window constitutes the display to indicate the set dose. Preferably, the sliding element and the sliding window are respectively configured such that the sliding element covers all indicia on the scale drum visible through the longitudinal window but one indicia on the scale drum, which corresponds to the set dose. For that purpose, the sliding element and the longitudinal window may be adapted such that when the sliding element slides axially in relation to the housing, the sliding element extends from a proximal end of the longitudinal window to a distal end of the longitudinal window such that the view on the scale drum through the longitudinal window is blocked wherein only though the sliding window, the one indicia corresponding to the set dose is visible. The sliding element may be axially movable from a position corresponding to a set dose of 0 units to a position corresponding to a maximum settable dose, wherein the sliding element is configured such that in both positions, the sliding element extends over the entire length of the longitudinal window in axial direction leaving only the sliding window through which one indicia, namely the indicia on the scale drum that corresponds to the set dose, is visible.

By providing the sliding window, a sleeve or other separate means to cover all indicia but the one that corresponds to the set dose is not necessary. In fact, through the longitudinal window, without the sliding element, the user would see a number of indicia, not knowing what the currently set dose is. The sliding element is configured to shield or cover all the indicia except the one that corresponds to the set dose. The indicia corresponding to the set dose is visible through the sliding window. The sliding element may be configured to have an extension that extends in the axial direction wherein the distal end and the proximal end of the sliding element is formed such that is does not collide with borders or edges of the window. For that purpose, the distal border of the longitudinal window may have a receiving section for receiving the extension such that the window of the sliding element can be placed over every single number on the scale drum.

The internal feature on the sliding element may be an internal thread feature such as a helical feature, preferably an internal male thread feature, such as a projection or the like engaging the external thread of the scale drum.

The sliding element may be provided with teeth or an axially extending splined portion configured to engage an axially extending groove on the inner surface of the housing. By such teeth/groove interface, the sliding element is rotationally constrained with respect to the housing but may move axially relative to the housing.

According to a further embodiment, the inner surface of the sliding element is in sliding contact with the outer surface of the scale drum between adjacent thread turns of the scale drum. Thereby, an improved support of the sliding element is provided. Blocking effects resulting from high friction are reduced.

According to a further embodiment, the drive spring is attached to a radially inner section of the scale drum. This effectively reduces the dimensions of the injection device, making it more compact.

A high degree of accuracy is achieved, when the drive spring is pre-wound or pre-charged upon assembly such that it applies a force or torque to the scale drum when the injection device is at zero units dialed. Thereby, when the user rotates the dial grip to set a dose, he rotates the number sleeve, and hence also the dose scale such that the drive spring is charged. This minimizes force or torque between components. By providing a minimum of force or torque, play between the components is effectively prevented.

The sliding element may be a shell-like component that at least partly extends circumferentially around the dose scale drum. The sliding element may have the form of a shield or strip extending in the longitudinal direction of the injection device. As an alternative, the sliding element may be at least partly formed as a sleeve. The sliding element may be used to shield or cover a portion of the indicia on the drum scale and to allow view only a limited portion of the drive scale.

In another embodiment, the sliding element extends about an angle of less than 360° in circumferential direction with respect to a longitudinal axis of the scale drum. The size of the device is further reduced. In other words, the sliding element does not surround the scale drum in a sleeve-like manner but only covers a section of the scale drum.

According to a further embodiment, the injection device comprises a trigger button or actuation button which the user may press to initiate dispense of a set dose of a liquid drug such as a medicament. The dial grip and the trigger button are rotationally fixed but axially movable relative to each other. Further, a clutch for releasably coupling the trigger button to the scale drum is provided by corresponding splined portions on the trigger button and the scale drum, wherein—preferably axial—movement of the trigger button from a first position into a second position causes the clutch to disengage. The trigger button and the dial grip may be rotationally fixed but axially movable relative to each other by means of a splined interface wherein the trigger button and the dial grip are provided with corresponding teeth and/or grooves that rotationally constrain the components to each other when engaged. By moving the trigger button from the first position into the second position, the splined interface is disconnected such that the scale drum may rotate relative to the trigger button. This mechanism provides for a convenient actuation of the device. The scale drum may be driven by the drive spring. Thus, when the user actuates the trigger button to dispense liquid medicament, the user disconnects the trigger button from the scale drum, and hence the actuation element is disconnected from the driving force.

According to a further embodiment, the trigger button is provided with splined features configured to engage corresponding splined features on the housing, wherein movement of the trigger button from the first position into the second position causes the splined features to engage such that the button is rotationally locked to the housing.

For safe and convenient dose setting, a further embodiment includes a drive sleeve and a clutch plate, wherein the clutch plate is rotationally constrained to the scale drum, e.g. by a splined interface preventing relative rotational movement between the clutch plate and the scale drum while allowing relative axial motion. The drive sleeve is movable from a first axial position to a second axial position relative to the housing and is configured to engage the housing in the first axial position such that the drive sleeve is rotationally constrained to the housing. For this purpose, the drive sleeve may be provided with a number of teeth on its outer surface that engage corresponding teeth and/or grooves on an inner surface of the housing when the drive sleeve is in the first axial position. The clutch plate is coupled to the drive sleeve via a ratchet interface such that the energy stored in the drive spring is prevented from being released when the drive sleeve is in the first position. The clutch plate may have a surface provided with angled teeth directly facing a surface of the drive sleeve that is provided with corresponding angled teeth. The injection device may further be provided with a clutch spring arranged such as to bias the clutch plate onto the drive sleeve. The angled teeth of the drive sleeve and the clutch plate may be arranged such that when the surfaces contact each other, relative rotation generates an audible click. In the direction of the spring torque, the torque can be transferred from the scale drum and the clutch plate to the drive sleeve.

According to a further embodiment, the drive sleeve is free to rotate relative to the housing in the second position. The drive sleeve and the housing may be provided with a splined interface configured such that when the drive sleeve is moved from the first into the second position, the splined interface disengages and the drive sleeve is free to rotate relative to the housing.

Preferably, the drive sleeve is rotationally constrained to a lead screw via a splined interface. When the drive sleeve is rotated, the lead screw is forced to move axially relative to the drive sleeve as the lead screw is threadedly engaged with the housing or a housing body. By rotation of the lead screw, the lead screw is displaced in the axial direction. The lead screw may be provided with a bearing at its distal end which bearing is in contact with a cartridge bung in a cartridge. By displacement of the lead screw in the distal direction, medicament in the cartridge is dispensed.

In order to initiate the dispense of the injection device, a further embodiment of the injection device is configured such that the trigger button displaces or moves the drive sleeve into the second axial position when the trigger button is moved from the first into the second position. The drive sleeve is further configured to engage the scale drum in the second position such that the drive sleeve is rotationally constrained to the scale drum. The drive sleeve and the scale drum may be provided with corresponding teeth and/or grooves to constitute a splined tooth interface. When the drive sleeve is moved into the second, preferably distal position, the drive sleeve disengages from its rotational lock with the housing and forms a rotational lock with the drum scale, so that the charged energy of the drive spring can be directly transferred from the drum scale to the drive sleeve.

In a further embodiment, the injection device comprises rotational stops defining a zero dose position and preferably also a maximum dose position. The rotational stops may be provided on the scale drum and a corresponding rotational stop may be provided on the sliding element. The rotational stops may be formed, e.g. as protrusions and/or abutments, preferably formed in the thread engagement between the scale drum and the sliding element.

In accordance with the further embodiment, the drive spring is a torsion spring. The torsion spring may be formed from a helical wire with at least two different pitches. In a central portion, the torsion spring may have open coils, meaning that the coils do not contact each other while adjacent coils at the ends of the torsion spring contact each other. The open coils allow the spring to compress to accommodate additional turns of wire without increasing the total length of the spring. Further, the open coils allow the spring to be compressed during assembly.

It has been proven effective, when the scale drum is provided with a receiving section configured to firmly receive an end of the spring configured as a hook, wherein the receiving section comprises a lead-in section and/or a groove section followed by an anchor point for the end of the drive spring. The incorporated lead-in is preferably large in diameter and the groove on the scale drum provides for automated assembly of the drive spring into the drum scale. As the drive spring is rotated during assembly, the hook-end form locates in the groove feature before engaging the anchor point in the drum scale. Further, a one-way clip feature may be provided that has to prevent the drive spring disengaging the anchor point during the assembly.

The housing may be a body like component that houses the scale drum. The body may also be a body element that is fixed to an outer housing or casing.

Preferably, the cartridge contains a liquid drug such as a medicament.

The term “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

### des Pro36 Exendin-4(1-39)-Lys6-NH2 (AVE0010),

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra-low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two 13 sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ, and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three on the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

### BRIEF DESCRIPTION OF THE DRAWINGS

Non-limiting, exemplary embodiments will now be described with reference to the accompanying drawings, in which:

FIG. 1 shows an exploded view of an injection device in accordance with a first embodiment;

FIG. 2 shows an exploded view of an injection device in accordance with a second embodiment;

FIG. 3 a perspective view of the sliding element of the device in FIG. 2;

FIG. 4 a perspective view of the number sleeve of the device in FIG. 2;

FIG. 5 a perspective view of another section of the number sleeve in FIG. 4;

FIG. 6 a perspective view of the drive spring of the device in FIG. 2;

FIGS. 7a,b perspective views of the button and the number sleeve of the device in FIG. 2;

FIG. 8 a perspective view of parts of the drive mechanism of the device in FIG. 2;

FIGS. 9a,b perspective views of the drive sleeve and the clutch plate of the device in FIG. 2;

FIGS. 10a,b a dose setting sequence of the device in FIG. 2 in a side view;

FIG. 11 a perspective view of the button and the housing of the device in FIG. 2;

FIG. 12 the device in FIG. 2 in a cut view; and

FIGS. 13a,b interaction between the drive sleeve and the number sleeve of the device in FIG. 2.

### DETAILED DESCRIPTION

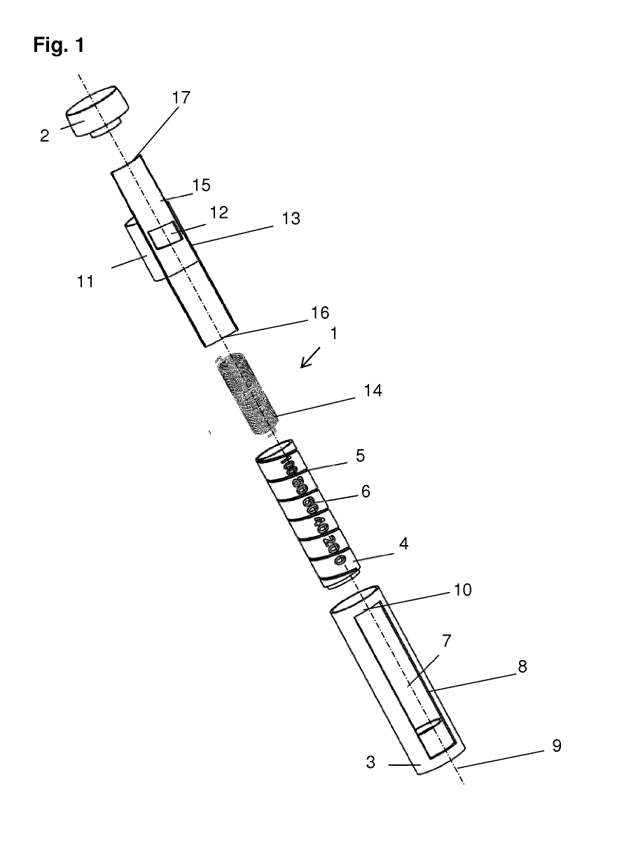

FIG. 1 shows an exploded view of a first embodiment of the injection device 1 with its components which are a dose dial 2 in the form of a dial button, a housing or body 3, a dose scale drum or number sleeve 4 which has an outer thread 5 on its outer peripheral surface extending in a helical pattern from a distal end to a proximal end. The scale drum 4 carries indicia 6 which are printed on the scale drum. The indicia 6 are helically provided on the scale drum 4.

The housing or body 3 has an elongated window or aperture 7 of rectangular shape with two longitudinal borders 8 extending parallel to the longitudinal axis 9 of the injection device and two radial borders 10 perpendicular to the longitudinal axis 9. Through the window 7, the user can inspect the drum scale 4.

The dose dial 2 is axially retained in the housing 3 and the scale drum 4 is directly coupled to the dial button 2 to follow the rotation of the dial button 2 such that when a user rotates the dial button 2 to select a dose, the scale drum 4 rotates together with the dial button 2. The dial button 2 and the scale drum 4 are arranged such that they both rotate without any axial displacement. The dose dial 2 also has the function of a dose or trigger button. The dial button 2 and the scale drum 4 are releasably coupled such that when the set dose is injected, the dial button 2 does not necessarily rotate back with the scale drum 4.

The external helical thread 5 of the scale drum 4 is engaged by a corresponding male thread of a sliding element 11. The sliding element 11 has a tubular section and a window or sliding window 12, wherein on two sides of the window 12 in axial direction, the male thread for engaging the helical thread 5 of the scale drum 4 is formed. In a further embodiment of the device shown, the inner surface of the sliding element 11 is in sliding contact with the outer surface of the scale drum between adjacent thread turns of the scale drum.

The inner surface of the housing 3 is provided with longitudinal bars that guide the sliding element 11 in axial direction but prevent relative rotation between the sliding element 11 and the housing 3. The longitudinal bars engage longitudinal recesses 13 on the outer surface of the sliding element 11. Due to this engagement in combination with the engagement between the threads of the housing 3 and the sliding element 11, the sliding element 11 moves axially whenever the scale drum 4 is rotated. The axial movement of the sliding element 11 and thus of the sliding window 12 relative to the longitudinal window 7 in the housing 3 is coordinated with the helical pattern of the indicia 6 printed on the scale drum 4 such that only one indicia 6 is present in the longitudinal window 7 and the sliding window 12 at the same time.

A drive spring 14 is connected to the scale drum 4 with one end and to the housing 3 with another end such that relative rotation between the scale drum 4 and the housing charges 3 the drive spring. The axial length of the sliding element 11 is sufficient to cover the visible part of the helical track 5 of the drum scale 4 in order to fully prevent the user from viewing the indicia 6 not in sight through the sliding window 12. For that purpose, the sliding element 11 has an extension 15 that extends in the axial direction wherein the distal end 16 and proximal end 17 of the sliding element 11 is formed such that is does not collide with the borders 10 of the window 7. For that purpose, the distal border 10 may have a receiving section for receiving the extension such that the window 12 of the sliding element 11 can be placed over every single number on the scale drum 4.

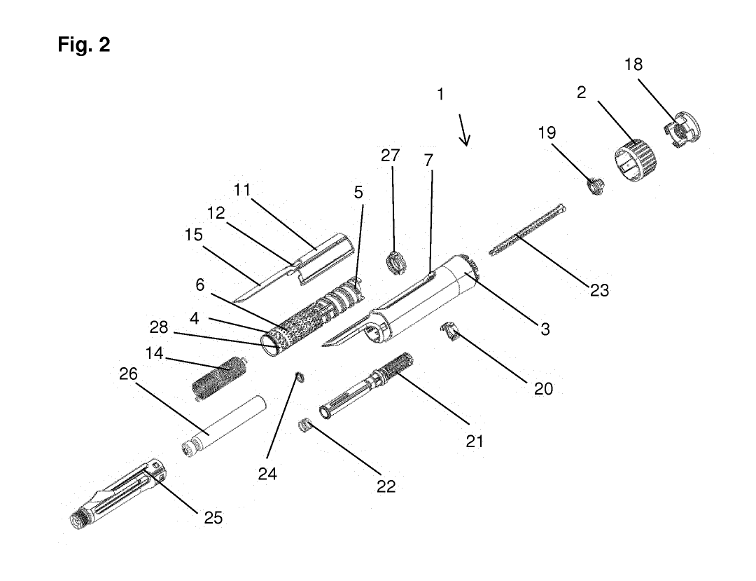

FIG. 2 shows an exploded view of the components of a further embodiment of the injection device.

The device 1 comprises a dose dial 2 in the form of a dial grip, a housing and/or housing body 3 with an elongated window 7, a dose scale drum in the form of a number sleeve 4 which has an outer thread 5 on its outer peripheral surface extending in a helical pattern from a distal end to a proximal end. The number sleeve 4 carries indicia 6 which are printed on the scale drum. The indicia 6 are on the scale drum 4 in a helical pattern. The device further comprises a trigger button 18, a sliding element 11 configured as a gauge component with a sliding window 12, a clutch plate 19, a last dose nut 20, a drive sleeve 21, a clutch spring 22, a lead screw 23, a bearing 24 provided at a distal end of the lead screw 23, a drive spring 14 in the form of a torsion spring, a cartridge holder 25 that can be attached to the distal end of the housing 3 and that receives a cartridge 26 which is filled with a medicament and which has a bung (not shown) inside wherein when the bearing 24 is moved in distal direction, the bearing displaces the bung such that medicament is dispensed from the cartridge when a dispense interface such as a double ended needle cannula is attached to the distal end of the cartridge. The number sleeve 4 comprises an upper number sleeve part 27 referred to a number sleeve upper and a lower number sleeve part 28 referred to as number sleeve lower. In contrast to the embodiment in FIG. 1, the dose dial 2 and the button 18 are separate individual components. All components are located concentrically about a common principal longitudinal axis of the mechanism. The body 3 may also be a body element that it fixed to an outer housing or casing.

The button 18 is permanently splined to the dose dial 2. It is also splined to the number sleeve upper 28 when the button 18 is not pressed, but this spline interface is disconnected when the button 18 is pressed. When the button 18 is pressed, splines on the button 18 engage with splines on the housing 3 preventing rotation of the button 18 (and hence the dose dial 2) during dispense.

These splines disengage when the button 18 is released, allowing a dose to be dialed. The dose dial 2 is axially constrained to the housing 3. It is rotationally constrained, via the splined interface to the button 18. The number sleeve lower 28 is rigidly fixed to the number sleeve upper 27 during assembly to form the number sleeve 4 and is a separate component to simplify number sleeve 4 mold tooling and assembly. This sub assembly is constrained to the housing 3 by holding elements (not shown) towards the distal end to allow rotation but not translation. The number sleeve lower 28 is marked with indices in the form of a sequence of numbers, which are visible through the window 12 of the sliding element 11 and the window 7 in the housing 3 to denote the dialed dose of medicament.

The clutch plate 19 is splined to the number sleeve 4. It is also coupled to the drive sleeve 21 via a ratchet interface. The ratchet provides a detented position between the number sleeve 4 and the drive sleeve 21 corresponding to each dose unit and engages different ramped tooth angles during clockwise and anti-clockwise relative rotation. The sliding element 11 is constrained to prevent rotation but allow translation relative to the housing 3 via a splined interface. The sliding element 11 has a helical feature on its inner surface which engages with the helical thread 5 cut in the number sleeve 4 such that rotation of the number sleeve 4 causes axial translation of the sliding element 11. This helical feature on the sliding element 11 also creates stop abutments against the end of the helical cut in the number sleeve 4 to limit the minimum and maximum dose that can be set.

The last dose nut 20 is located between the number sleeve 4 and the drive sleeve 21. It is rotationally constrained to the number sleeve 4 via a splined interface. It moves along a helical path relative to the drive sleeve 21 via a threaded interface when relative rotation occurs between the number sleeve 4 and drive sleeve 21. The drive sleeve 21 extends from the interface with the clutch plate 19 to the contact with the clutch spring 22. A splined tooth interface with the number sleeve 4 is not engaged during dialing, but engages when the button 18 is pressed, preventing relative rotation between the drive sleeve 21 and number sleeve 4 during dispense. A further splined tooth interface with the housing 3 prevents rotation of the drive sleeve 21 during dose setting. When the button 18 is pressed, the drive sleeve 21 and the housing 3 disengage allowing the drive sleeve 21 to rotate.

The helical drive spring 14 is charged and stores energy during dose setting by the action of the user rotating the dose dial 2. The spring energy is stored until the mechanism is triggered for dispense at which point the energy stored is used to deliver the medicament from the cartridge to the user. The drive spring 14 is attached at one end to the housing 3 and at the other end to the number sleeve 4. The drive spring 14 is pre-wound upon assembly, such that it applies a torque to the number sleeve 4 when the mechanism is at zero units dialed. The action of rotating the dose dial 2 to set a dose, rotates the number sleeve 4 relative to the housing 3 and charges the drive spring 14 further.

The lead screw 23 is rotationally constrained to the drive sleeve 21 via a splined interface. When rotated, the lead screw 23 is forced to move axially relative to the drive sleeve 21, through a threaded interface with the housing 3 (not shown). The bearing 24 is axially constrained to the lead screw 23 and acts on a bung within the liquid medicament cartridge 26.

The axial position of the drive sleeve 21, clutch plate 19 and button 18 is defined by the action of the clutch spring 22, which applies a force on the drive sleeve 21 in the proximal direction. This spring force is reacted via the drive sleeve 21, clutch plate 19 and button 18, and when ‘at rest’ it is further reacted through the dose dial 2 to the housing 3. The spring force ensures that the ratchet interface is always engaged. In the ‘at rest’ position, it also ensures that the button splines are engaged with the number sleeve 4 and that the drive sleeve teeth are engaged with the housing 3. The housing 3 provides location for the liquid medication cartridge and cartridge holder 25, windows for viewing the dose number and the sliding element, and a feature on its external surface to axially retain the dose dial 2 (not shown). A removable cap fits over the cartridge holder 25 and is retained via clip features on the housing 3.

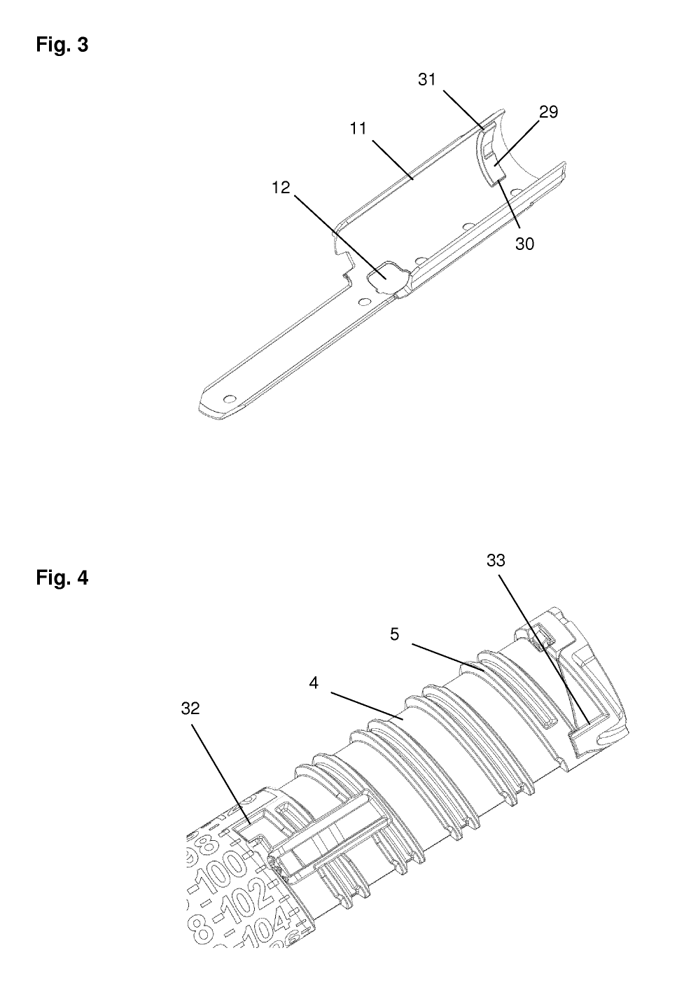

FIG. 3 shows the inside of the sliding element 11 with the window 12 and the male thread feature 29 on the inner surface of the sliding element 11 that engages the outer thread 5 on the number sleeve 4 (see FIG. 4). The thread feature 29 has a zero dose abutment 30 and a maximum dose abutment 31. As shown in FIG. 4, the outer thread 5 has a zero dose abutment 32 at one end of the thread 5 and a maximum dose abutment 33 at the other end of the thread 5 so that any dose size can be selected between zero and a pre-defined maximum, in increments to suit the medicament and user profile. The drive spring 14, which has a number of pre-wound turns applied to it during assembly of the device, applies a torque to the number sleeve 4 and is prevented from rotating by the zero dose abutment.

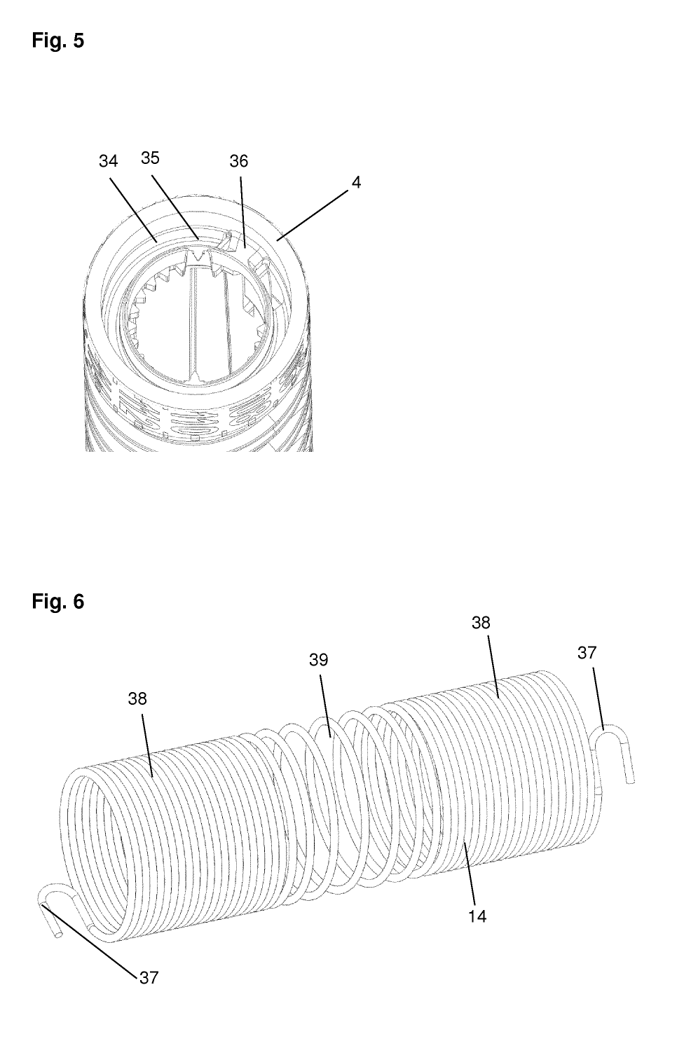

As shown in FIG. 5, the inner surface of the number sleeve 4 has a lead-in 34 followed by a groove 35 and an anchor point 36. Automated assembly of the drive spring 14 into the number sleeve is achieved by incorporating the large lead-in 34 and the groove feature 35. As the drive spring 14 is rotated during assembly, a hook end form 37 at the one end of the drive spring 14 (see FIG. 6) is located in the groove feature 35 before engaging the anchor point 36 in the number sleeve 4.

As shown in FIG. 6, the drive spring 14 is formed from a helical wire with at least two different pitches. Both ends are formed from ‘closed’ coils 38, i.e. the pitch equals the wire diameter and each coil contacts the adjacent coil. The central portion has ‘open’ coils 39, i.e. the coils do not contact each other. This has the following advantages. When a dose is set, the drive spring 14 is charged. If all the coils were closed, winding up the spring would increase the length of the spring by one wire diameter for each turn, and so the hook ends would no longer be aligned with their anchor points on the housing and number sleeve. The open coils allow the spring to compress to accommodate the additional turns of wire, without increasing the total length of the spring. Further, the open coils 39 allow the spring to be compressed during assembly. The spring is manufactured longer than the space available in the device. It is then compressed during assembly, ensuring that the axial positions of the hook ends are better aligned with their anchor points on the housing and the number sleeve. Also, it is easier to manufacture the spring to a specified length if most of the coils are closed, as the length of these coils is only a function of the wire diameter. Moreover, following assembly, compression in the spring biases the number sleeve axially relative to the housing in a consistent direction, reducing the effects of geometric tolerances. Further, the addition of closed coils at each end makes the springs less prone to tangling with each other when they are stored together between manufacture and assembly and closed coils at the ends provide a flat surface for contact with the housing and the number sleeve.

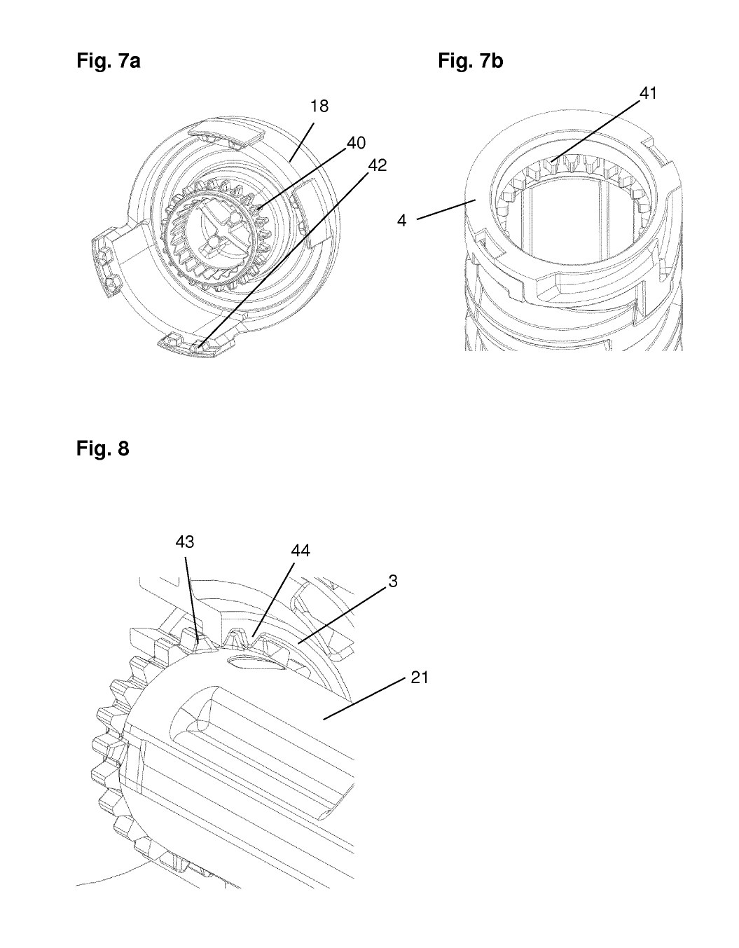

For selecting a dose, the user rotates the dial grip 2 clockwise. As shown in FIGS. 7a and 7b, the button has inner splines 40 for engaging corresponding splines 41 on the upper part of number sleeve 4 to create a splined interface 40/41. The dial grip is splined to the button 18, wherein the button 18 has a further set of splines 42 for engagement with corresponding splines of the housing 3. During dose selection, rotation of the dial grip is transferred to the button 18. The button 18 is in turn splined to the upper number sleeve (during dose selection only) via the splines 40. The upper number sleeve is permanently fixed to the lower number sleeve to form the number sleeve 4. Therefore, rotation of the dial grip 2 generates an identical rotation in the number sleeve 4. Rotation of the number sleeve 4 causes charging of the drive spring, increasing the energy stored within it. As the number sleeve 4 rotates, the sliding element 11 translates axially due to its threaded engagement with the number sleeve 4 thereby showing the value of the dialed dose.

As shown in FIG. 8, the drive sleeve 21 has splines 43 for engaging corresponding splines 44 formed on the inside of the housing 3 to create a splined interface 43/44. The drive sleeve 21 is prevented from rotating as the dose is set and the number sleeve is rotated, due to the engagement of its splined teeth 43 with the teeth 44 of the housing 3. Relative rotation therefore occurs between the clutch plate that is driven by the number sleeve and the drive sleeve via the ratchet interface.

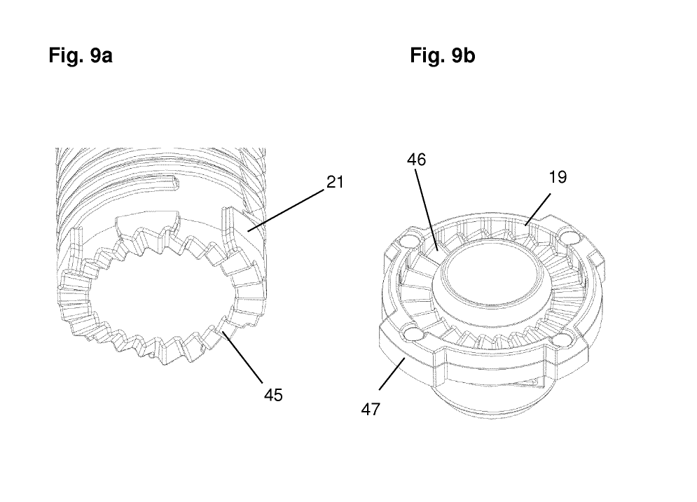

As shown in FIGS. 9a and 9b, an end surface of the drive sleeve 21 is provided with angled teeth 45 to form a ratchet interface 45/46 with angled teeth 46 on the clutch plate 19. On the outer circumference of the clutch plate 19, splined teeth 47 for engaging a corresponding groove on the number sleeve are formed. The user torque required to rotate the dial grip is a sum of the torque required to wind up the drive spring, and the torque required to overhaul the ratchet feature 45/46. The clutch spring is designed to provide an axial force to the ratchet feature 45/46 and to bias the clutch plate 19 onto the drive sleeve 21. This axial load acts to maintain the ratchet teeth engagement of the clutch plate 19 and the drive sleeve 21. The torque required to overhaul the ratchet in the dose set direction is a function of the axial load applied by the clutch spring, the clockwise ramp angle of the ratchet, the friction coefficient between the mating surfaces and the mean radius of the ratchet features. As the user rotates the dial grip sufficiently to increment the mechanism by 1 increment, the number sleeve 14 rotates relative to the drive sleeve 21 by 1 ratchet tooth. At this point the ratchet teeth re-engage into the next detented position. An audible click is generated by the ratchet re-engagement, and tactile feedback is given by the change in torque input required.

With no user torque applied to the dial grip 21, the number sleeve 4 is prevented from rotating back under the torque applied by the drive spring 14, due solely to the ratchet engagement 45/46 between the clutch plate 19 and the drive sleeve 21. The torque necessary to overhaul the ratchet in the anti-clockwise direction is a function of the axial load applied by the clutch spring 22, the anti-clockwise ramp angle of the ratchet 45/46, the friction coefficient between the mating surfaces and the mean radius of the ratchet features. The torque necessary to overhaul the ratchet must be greater than the torque applied to the number sleeve 4 (and hence clutch plate 19) by the drive spring 14. The ratchet ramp angle is therefore increased in the anti-clockwise direction to ensure this is the case whilst ensuring the dial-up torque is as low as possible.

The user may choose to increase the selected dose by continuing to rotate the dial grip in the clockwise direction. The process of overhauling the ratchet interfaces between the number sleeve 4 and drive sleeve 21 is repeated for each dose increment. Additional energy is stored within the drive spring 14 for each dose increment and audible and tactile feedback is provided for each increment dialled by the re-engagement of the ratchet teeth. The torque required to rotate the dial grip 2 increases as the torque required to wind up the drive spring 14 increases. The torque required to overhaul the ratchet in the anti-clockwise direction must therefore be greater than the torque applied to the number sleeve 4 by the drive spring 14 when the maximum dose has been reached.

If the user continues to increase the selected dose until the maximum dose limit is reached, the number sleeve 4 engages with its maximum dose abutment on the sliding element (see FIGS. 3 and 4). This prevents further rotation of the number sleeve 4, clutch plate 19 and dial grip 2.

The last dose nut is splined to the number sleeve while the last dose nut is threaded to the drive sleeve such that relative rotation of the number sleeve and the drive sleeve during dose setting also causes the last dose nut to travel along its threaded path towards a last dose abutment on the drive sleeve. Depending on how many increments have already been delivered by the mechanism, during selection of a dose, the last dose nut may contact its last dose abutment with the drive sleeve. The abutment prevents further relative rotation between the number sleeve 4 and the drive sleeve 21 and therefore limits the dose that can be selected. The position of the last dose nut is determined by the total number of relative rotations between the number sleeve 4 and the drive sleeve 21, which have occurred each time the user sets a dose.

When a dose has been set, the user is able to deselect any number of increments from this dose. Deselecting a dose is achieved by the user rotating the dial grip 2 anti-clockwise. The torque applied to the dial grip 2 by the user is sufficient, when combined with the torque applied by the drive spring 14, to overhaul the ratchet between the clutch plate 19 and the drive sleeve 21 in the anti-clockwise direction. When the ratchet 45/46 is overhauled, anti-clockwise rotation occurs in the number sleeve 4 (via the clutch plate 19), which returns the number sleeve 4 towards the zero dose position, and unwinds the drive spring 14. The relative rotation between the number sleeve 4 and drive sleeve 21 causes the last dose nut to return along its helical path, away from the last dose abutment.

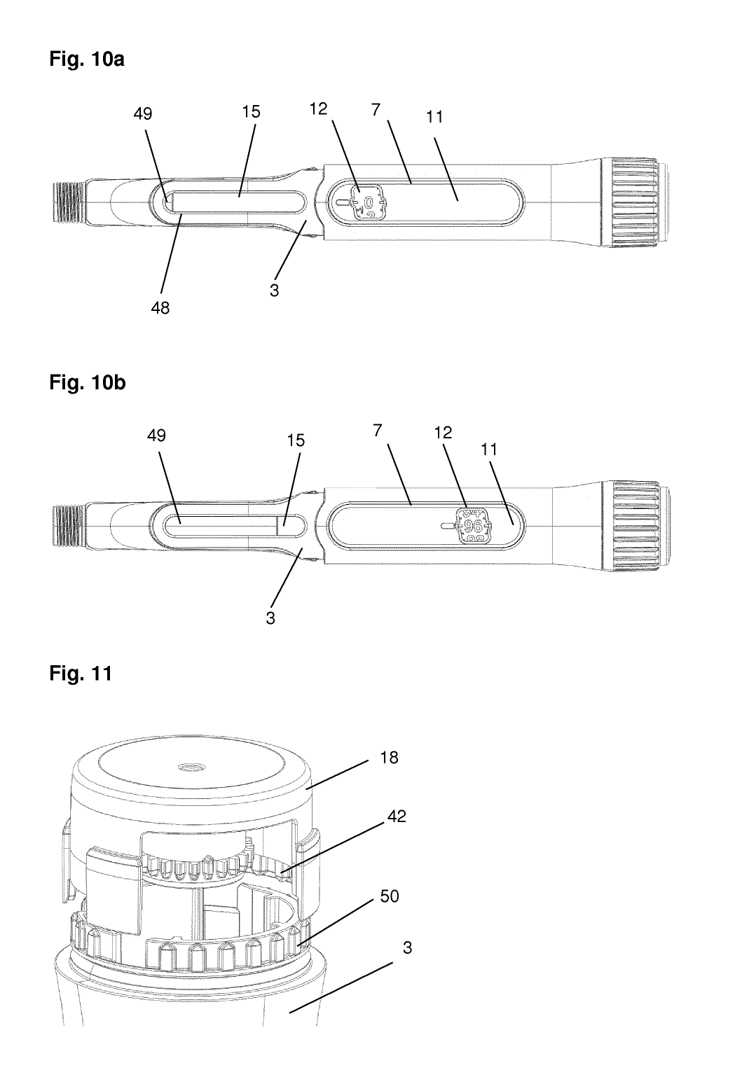

As shown in FIGS. 10a and 10b, the sliding element 11 has flanges or extensions on either side of the window area which cover the numbers printed on the number sleeve adjacent to the dialed dose to ensure only the set dose number is made visible to the user. The device includes a visual feedback feature in addition to the discrete dose number display typical on devices of this type. The distal end of the sliding element 11 has the extension 15 (see FIG. 2) that creates a sliding scale through a small window 48 in the housing 3. As a dose is set by the user, the sliding element 11 translates axially, the distance moved proportional to the magnitude of the dose set. This feature gives clear feedback to the user regarding the approximate size of the dose set. The dispense speed of an auto-injector mechanism may be higher than for a manual injector device, so it may not be possible to read the numerical dose display during dispense. The sliding element 11 provides feedback to the user during dispense regarding dispense progress without the need to read the dose number itself.

The window 48 may be formed by an opaque element on the sliding element 11 revealing a contrasting colored component 49 underneath. Alternatively, the revealable element 49 may be printed with coarse dose numbers or other indices to provide more precise resolution. In addition, this display simulates a syringe action during dose set and dispense.

To reduce dust ingress and prevent the user from touching moving parts, the viewing openings 7 and 48 in the housing 3 are covered by translucent windows. These windows may be separate components, but in this embodiment they are incorporated into the housing 3 using ‘twin-shot’ molding technology. A first shot of translucent material forms the internal features and the windows, and then a ‘second shot’ of opaque material forms the outer cover of the housing 3.

Delivery of a dose is initiated by the user depressing the button axially. When the button 18 (see FIGS. 7a and 7b) is depressed, the splines 40 and 41 between the button 18 and the number sleeve 4 disengage, rotationally disconnecting the button 18 and dial grip 21 from the delivery mechanism.

As shown in FIG. 11, the splines 42 on the button 18 engage with splines 50 on the housing 3 preventing rotation of the button 18 (and hence the dial grip 21) during dispense. As the button 18 is stationary during dispense, it can be used in a dispense clicker mechanism. A stop feature in the housing 3 limits axial travel of the button 18 and reacts any axial abuse loads applied by the user, reducing the risk of damaging internal components.

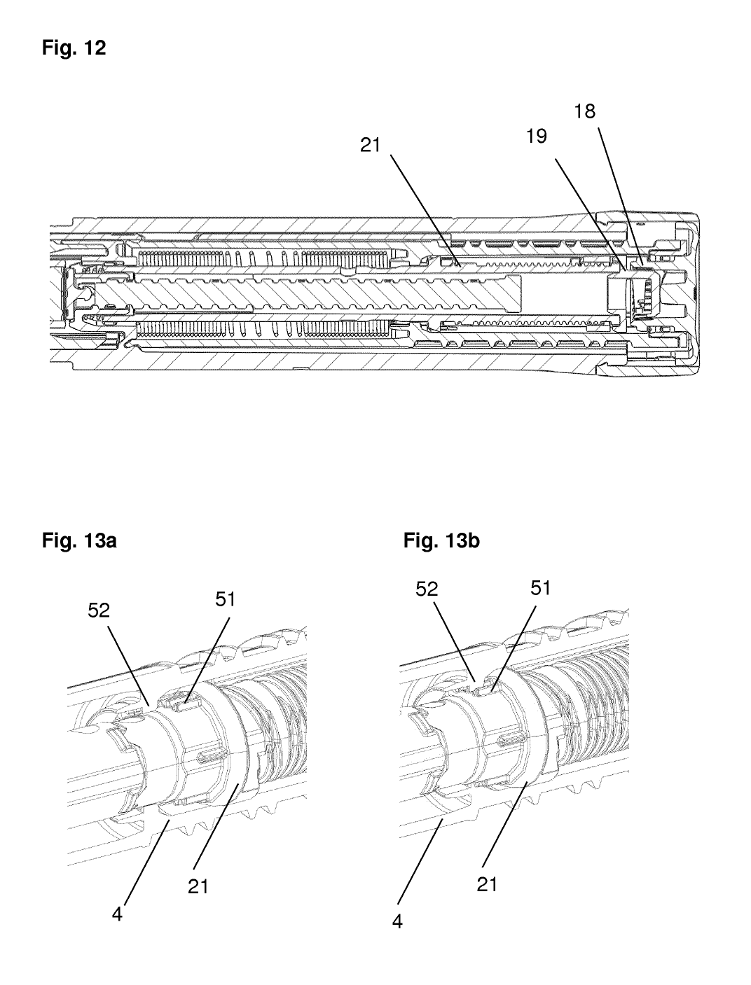

As shown in FIG. 12, the clutch plate 19 arranged between the drive sleeve 21 and the button 18 is moved axially by the button and the drive sleeve 21 is moved axially by the clutch plate 19.

As shown in FIGS. 13a and 13b, the axial displacement of the drive sleeve 21 engages splines 51 on the drive sleeve 21 with splines 52 on the number sleeve 4 so that a splined tooth interface 51/52 is formed preventing relative rotation between the drive sleeve 21 and number sleeve 4 during dispense. The splined tooth interface 43/44 (FIG. 8) between the drive sleeve 21 and the housing 3 disengages, so that the drive sleeve 21 can now rotate relative to the housing 3 and is driven by the drive spring via the number sleeve 4, and clutch plate 19. Rotation of the drive sleeve 21 causes the lead screw 23 to rotate due to their splined engagement, and the lead screw 23 then advances due to its threaded engagement to the housing 3. The number sleeve 4 rotation also causes the sliding element to traverse axially back to its zero position whereby the zero dose abutment (FIG. 3 and FIG. 4) stops the mechanism.

It is possible to angle the spline teeth on either the drive sleeve 21 or the housing 3, so that when the zero dose abutment 30 stops rotation of the number sleeve 4 and hence the drive sleeve 21 at the end of the dose and the button 18 is released the spline teeth between the drive sleeve 21 and the housing 3 rotate the drive sleeve 21 backwards by a small amount and hence move the lead screw 23 axially back away from the bung and rotates the number sleeve lower 28 from the zero dose stop position. This helps to prevent possible weepage.

### REFERENCE NUMERALS

1 injection device (drug delivery device)
2 dose dial/dial grip
3 housing/body
4 dose scale drum/number sleeve
5 outer thread
6 indicia
7 window
8 longitudinal border
9 longitudinal axis
10 radial border
11 sliding element
12 sliding window
13 recess
14 drive spring
15 extension
16 distal end of sliding element
17 proximal end of sliding element
18 trigger button
19 clutch plate
20 last dose nut
21 drive sleeve
22 clutch spring
23 lead screw
24 bearing
25 cartridge holder
26 cartridge
27 upper number sleeve part
28 lower number sleeve part
29 male thread feature
30 zero dose abutment of sliding element
31 maximum dose abutment of sliding element
32 zero dose abutment of number sleeve
33 maximum dose abutment of number sleeve
34 lead-in
35 groove
36 anchor
37 hook
38 closed coils
39 open coils
40 splines of button
41 splines of number sleeve
42 splines of button
43 splines of drive sleeve
44 splines of body
45 angled teeth
46 angled teeth
47 splines of clutch plate
48 window
49 revealable element
50 splines on body
51 splines on drive sleeve
52 splines on number sleeve

1-17. (canceled)

18. An injection device for automatic spring driven injection of a liquid drug by which doses of an individual size can be set by a user, the injection device comprising:
a housing defining an interior space and having a longitudinal window,
a rotatable dose dial axially retained in relation to the housing,
a rotatable scale drum carrying indicia for indicating a size of a set dose, wherein the scale drum is functionally coupled to the dose dial to rotate when the dose dial is rotated to set a dose,
a sliding element provided with a sliding window, wherein the sliding element is adapted to slide axially in relation to the housing during dose setting, and the indicia carried by the scale drum is visible through the sliding window such that the longitudinal window and the sliding window in combination with the indicia form a dose size display,
wherein the scale drum rotates within an interior space defined by the housing during dose setting, an inner surface of the sliding element is provided with an internal feature engaging an external thread provided on an outer surface of the scale drum, and the sliding element is further axially guided in the housing such that the sliding element moves axially when the scale drum is rotated, and
wherein a drive spring is attached to the scale drum with one end and to the housing with another end such that relative rotation between the scale drum and the housing charges the drive spring.

19. The injection device according to claim 18, wherein the inner surface of the sliding element is in sliding contact with the outer surface of the scale drum between adjacent thread turns of the scale drum.

20. The injection device according to claim 18, wherein the drive spring is attached to a radially inner section of the scale drum.

21. The injection device according to claim 18, wherein the sliding element and the sliding window are respectively configured such that the sliding element covers all indicia on the scale drum visible through the longitudinal window except one indicia on the scale drum, which corresponds to the set dose.

22. The injection device according to claim 18, wherein the drive spring is pre-wound upon assembly such that it applies a force or torque to the scale drum when the injection device is at zero units dialed.

23. The injection device according to claim 18, wherein the sliding element is a shell-like component that at least partly extends circumferentially around the scale drum.

24. The injection device according to claim 23, wherein the sliding element extends about an angle of less than 360° in a circumferential direction with respect to a longitudinal axial of the scale drum.

25. The injection device according to claim 18, wherein the sliding element is axially movable from a position corresponding to a set dose of zero units to a position corresponding to a maximum settable dose, wherein the sliding element is configured such that in both positions, the sliding element extends over the entire length of the longitudinal window in axial direction, and the one indicia that corresponds to the set dose is only visible through the sliding window.

26. The injection device according to claim 18, comprising a trigger button, wherein the trigger button and the dial grip are rotationally fixed and axially movable relative to each other, and wherein a releasable clutch for releasably coupling the trigger button to the scale drum is provided by corresponding splined portions on the trigger button and the scale drum, wherein movement of the trigger button from a first position into a second position causes the clutch to disengage.

27. The injection device according to claim 26, wherein the trigger button is provided with spline features configured to engage corresponding spline features on the housing, wherein movement of the trigger button from the first position into the second position causes the spline features of the trigger button and the housing to engage such that the trigger button is rotationally locked to the housing.

28. The injection device according to claim 18, comprising a drive sleeve and a clutch plate wherein the clutch plate is rotationally constrained to the scale drum, wherein the drive sleeve is movable from a first axial position to a second axial position,
wherein the drive sleeve is configured to engage the housing in the first axial position such that the drive sleeve is rotationally constrained to the housing, and
wherein the clutch plate is coupled to the drive sleeve via a ratchet interface such that energy stored in the drive spring is prevented from being released when the drive sleeve is in the first position.

29. The injection device according to claim 28, wherein in the second position, the drive sleeve is free to rotate relative to the housing,

30. The injection device according to claim 28, wherein the shift between the first axial position of the drive sleeve and the second axial position of the drive sleeve is initiated by a trigger button moving from a first position of the trigger button to a second position of the trigger button.

31. The injection device according to claim 30, wherein the trigger button moves the drive sleeve into the second axial position of the drive sleeve when the trigger button is moved from the first position of the trigger button into the second position of the trigger button, wherein the drive sleeve is configured to engage the drum scale in the second axial position of the drive sleeve in such way that the drive sleeve is rotationally constrained to the drum scale.

32. The injection device according to claim 18, comprising corresponding rotational stops on the scale drum and the sliding element.

33. The injection device according to claim 18, wherein the drive spring is a torsion spring.

34. The injection device according to claim 18, wherein the scale drum is provided with a receiving section configured to firmly receive an end of the drive spring configured as a hook, wherein the receiving section comprises a lead-in section or a groove section followed by an anchor point for the end of the drive spring.

35. The injection device according to claim 18 further comprising a cartridge containing a liquid drug.
